(19) United States
(12) Patent Application Publication
(10) Pub. No.: US 2004/0031493 A1
(43) Pub. Date: Feb. 19, 2004

(54) Method for treating primary glaucoma caused by spasm of the meridional fibers

(76) Inventor: Vadim Osipov, Brighton, MA (US)

Correspondence Address: ALTMAN & MARTIN, 6 BEACON ST, STE 600, BOSTON, MA, 02108, US

(21) Appl. No.: 10/614,437

(22) Filed: Jul. 7, 2003

(51) Int. Cl. A61B 19/00

(52) U.S. Cl. 128/898

(57) ABSTRACT

A treatment for primary glaucoma caused by spasming of the medidional fibers resulting from presbyopia, including resection of the meridional fibers. The resection is preferably performed in front of the ora serrata, through the sclera, and between the places of attachment of the rectus muscles. The resection is preferably performed as a series of regularly spaced cuts in two parallel lines. Use of a surgical knife with a diamond blade is preferred.

### CROSS-REFERENCES TO RELATED APPLICATIONS

Not Applicable

### STATEMENT REGARDING FEDERALLY SPONSORED RESEARCH OR DEVELOPMENT

Not Applicable

### REFERENCE TO A SEQUENCE LISTING, A TABLE, OR A COMPUTER PROGRAM LISTING COMPACT DISK APPENDIX

Not Applicable

### BACKGROUND OF THE INVENTION

1. Field of the Invention

The present invention relates to glaucoma, more particularly, to a surgical treatment for the alleviation of primary glaucoma.

2. Description of the Related Art

About two percent of people in the United States have glaucoma. Between 80,000 and 116,000 Americans are legally blind as a result of glaucoma, and it is estimated that each year an additional 5,500 people become legally blind in the United States. As a consequence, glaucoma is a very important public health problem. Glaucoma is a progressive disease of the eye, characterized by increased pressure within the eyeball, damage to the optic disc, and loss of vision.

It is customary to consider that glaucoma begins when circulation of aqueous humor is infringed. Elevated intraocular pressure most often is a result of increased resistance to the outflow of aqueous humor from the eye. Presently there are no convincing arguments regarding beginning of primary glaucoma with normal intraocular pressure.

In one existing form of primary glaucoma, called normal-tension glaucoma (formerly, low-tension glaucoma), cupping of the optic nerve occurs despite intraocular pressure in the normal range of 10 to 21 mm Hg. Some studies have indicated that about one-third of all incidents of glaucoma are normal-tension glaucoma.

It is believed that causes of primary glaucoma include low blood pressure, autoimmune disease, anemia, heart arrhythmia, and thick blood. Thick blood has a harder time getting through the small blood vessels in the optic nerve to nourish it. Sleep apnea is getting increasing attention as a possible cause. Other causal theories include a diminished blood supply to the optic nerve that eventually destroys the nerve fibers, hemorrhages of the optic nerve disc, myopia, high blood pressure, vascular disease, migraines, and possibly transient high intraocular pressures during different times of the day and night.

There is some speculation that normal-tension glaucoma is a different disease from other types of glaucoma. At present, however, the principles of treatment for normal tension glaucoma are the same as for other types of glaucoma. Since pressure is still largely considered one of the major causes, it is recommended that intraocular pressure be reduced even further, to the low teens or even the high single digits, between 8 and 12 to 14 mm Hg, using medications alone or in combination, and, if necessary, laser techniques and/or surgery.

### BRIEF SUMMARY OF THE INVENTION

An object of the present invention is to provide a method for treating primary glaucoma based on a proposed cause thereof.

It is proposed herein that a cause of primary glaucoma is associated with the process of accomodation. Increased density and decreased elasticity of the lens caused by presbyopia causes difficulty in the functioning of the circular and meridional fibers of the ciliary body that control the lens. Decreased elasticity of the lens results in increased load on the meridional fibers, leading to their over irritation and spasming. This spasming causes the meridional fibers to contantly pull on the peripheral section of the anterior choroid, keeping that part of the choroid constantly abnormally thin with a commensurate reduction in blood flow. In the posterior segment of the eye, increased blood filling of the veins is followed by venous stasis and hemostasis of various blood vessels. Hemostatis causes edema and ischemia, leading to atrophy of the optic nerve and the formation of glaucomatous cupping.

Also, blood filling of the veins of the anterior portion of the eye increases, leading to venous stasis in the ciliary body and iris, altering the permeability of the blood vessels in the ciliary body. Possibly, the fluidity, viscosity, and the biochemical composition of the aqueous humor changes, resulting in increased resistance to the routes of aqueous humor outflow, and elevating intraocular pressure. When the blood-aqueous barrier and mechanism of autoregulation are preserved and damage of routes of outflow does not occur, primary glaucoma with normal intraocular pressure develops.

The proposed treatment of the present invention includes resection of the meridional fibers. The resection can be performed anywhere along the meridional fibers but is preferably performed in front of the ora serrata, through the sclera, and between the places of attachment of the rectus muscles. In order to prevent leakage of the vitreous humor, it is preferred that the resection be performed as a series of regularly spaced cuts in two parallel lines. The cuts of the two lines should overlap slightly to ensure that all of the meridional fibers 16 are resected. Use of a surgical knife with a diamond blade is recommended so that the vessels and nerves that pass through suprachoroidal spaces will regenerate if cut.

Other objects of the present invention will become apparent in light of the following drawings and detailed description of the invention.

### BRIEF DESCRIPTION OF THE DRAWINGS

For a fuller understanding of the nature and object of the present invention, reference is made to the accompanying drawings, wherein:

FIG. 1 is a cross sectional view of an eye;

FIG. 2 is a cross sectional view of a portion of the eye showing the normal blood supply of the eyeball;

FIG. 3 is a cross sectional view of a portion of the eye showing how spasm of the meridional fibers affects blood flow;

FIG. 4 is a side view in partial phantom showing where the meridional fibers are resected for the treatment of the present invention;

FIG. 5 is a front cross sectional view of the eye of FIG. 4 taken at the line 5-5 showing where the meridional fibers are resected for the treatment of the present invention; and

FIG. 6 shows how the meridional fibers are resected in a non-continuous fashion.

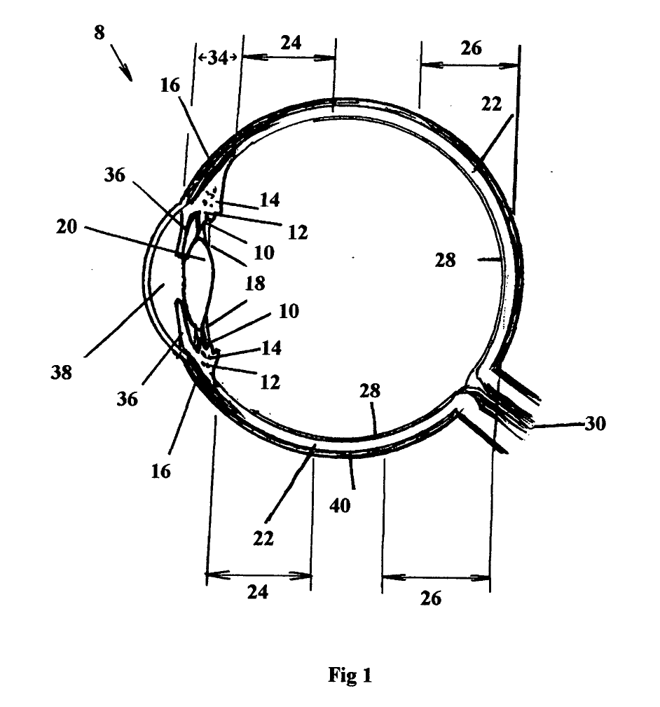

### DETAILED DESCRIPTION OF THE INVENTION

Accommodation is the process by which the eye 8 adjusts to near and far vision. The mechanism of accommodation is not fully understood. It is believed that when the eye 8 is accommodated for far vision, the circular fibers 14 of the ciliary muscle 12 are relaxed, increasing the inside diameter of the ciliary body 10. As a result, the zonule fibers 18 pull the edges of the lens 20 towards the ciliary body 10 and into a flattened shape. When the eye 8 is accommodated for near vision, the circular fibers 14 of the ciliary muscle 12 contract, releasing tension on the zonule fibers 18, allowing the lens 20 to shorten and thicken.

It is proposed herein that one cause for the onset and development of glaucoma is associated with the process of accomodation. It has been observed that the optic axis length changes in the process of accomodation. It is known that as the meridional fibers 16 of the ciliary body 10 contract, they pull the choroid 22, which is why this muscle is call m. tensor choridodea and also known as Brucke's muscle. However, the reason for this physiological function of meridional fibers 16 is unknown. It is proposed herein that meridional fibers 16 take part in the act of accommodation in the normal eye as follows. As the meridional fibers 16 contract, they pull the peripheral section of the anterior choroid 24, causing it to thin with a commensurate reduction in blood flow. Correspondingly, the blood flow in the central section of the posterior choroid 26 increases, causing that section to thicken. As a result, the retina 28 adjacent to the central section of the posterior choroid 26 moves forward, shortening the optical axis. The opposite result occurs when the meridional fibers 16 relax.

Increased density and decreased elasticity of the lens 20, typically a consequence of aging, causes presbyopia, the reduction in the range of accommodation. Increased density and decreased elasticity of the lens 20 also causes difficulty in the functioning of the circular and radial fibers of the ciliary body 10. When the circular fibers are relaxed, the lens 20 is stretched. Decreased elasticity of the lens 20 means that it cannot stretch as much. In compensation, the load on the meridional fibers 16 increases, and the consequent over irritation of the meridional fibers 16 can cause them to spasm.

It is further proposed herein that spasming of the meridional fibers 16 is a cause of primary glaucoma. Constant pulling of the meridional fibers 16 on the peripheral section of the anterior choroid 24 keeps that part of the choroid constantly abnormally thin with a commensurate reduction in blood flow.

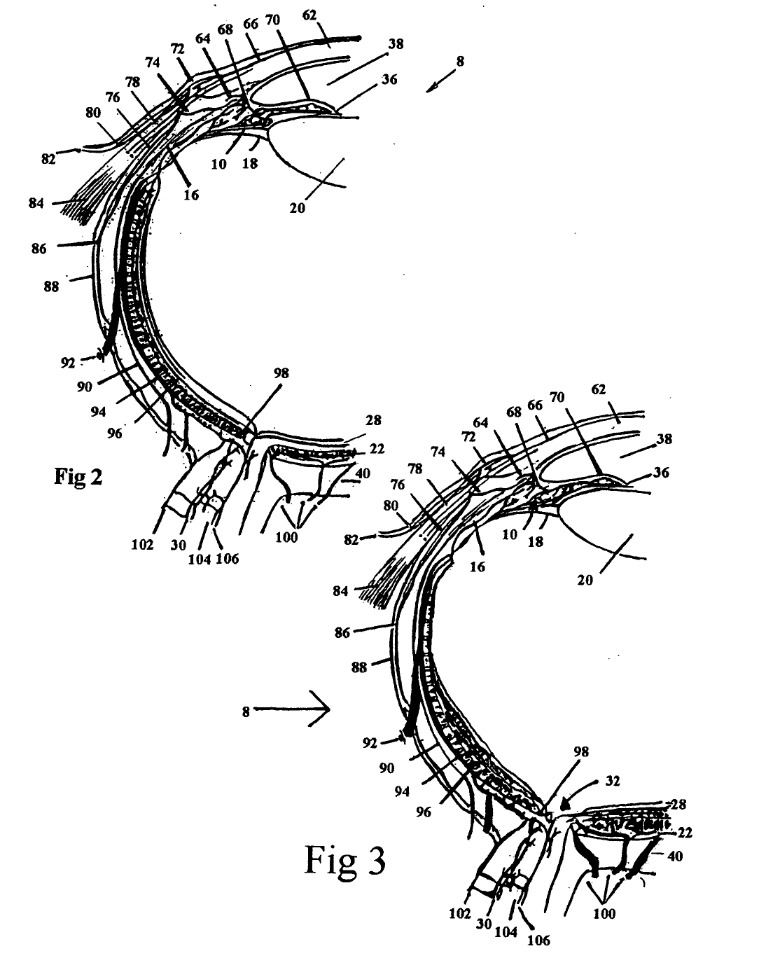

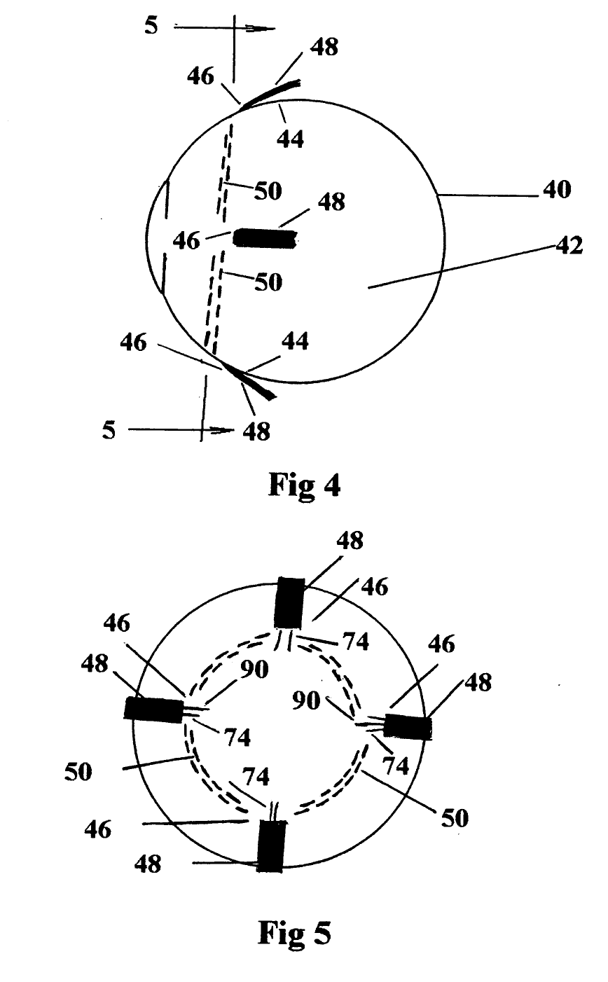

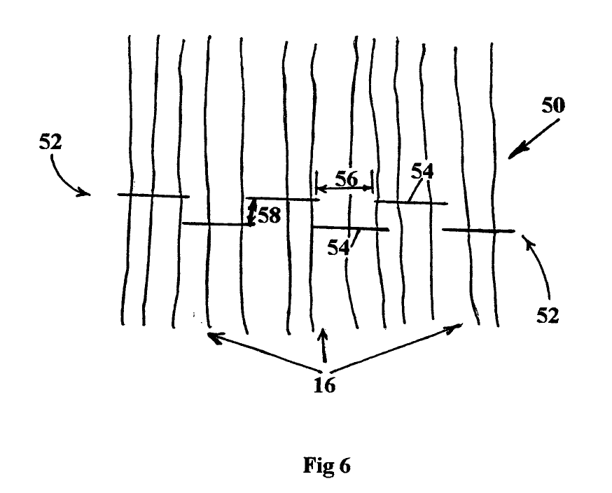

FIGS. 2 and 3 show the components of the eye 8, including arteries and veins affected by the spasming of the meridional fibers 16: the ciliary body 10, meridional fibers 16, zonule fibers 18, lens 20, choroid 22, retina 28, iris 36, aqueous humor 38, the cornea 62, Schlemm's canal 64, limbus 66; major arterial circle 68, minor arterial circle 70, anterior conjunctival arteries (veins) 72, anterior ciliary artery 74, recurrent artery 76, anterior ciliary vein 78, anterior ciliary artery 80, Posterior conjunctival artery and vein 82, medial rectus 84, apiscleral artery 86, apiscleral vein 88, long posterior ciliary artery 90, vena vorticosa 92 choriocapillaris 94, apteriole and venule retina 96, circle of Zinn-Haller 98, short posterior ciliary arteries 100, pial vessels 102, central artery 104, central vein 106, and sclera 40.

In the posterior segment of the eye 8, blood filling of the veins increases. This is followed by venous stasis and hemostasis (stagnation of blood) of the short posterior ciliary arteries 100 and their branches, the arteries of the prelaminar region (the arteries of the circle of Zinn-Haller 98), and the vessels of the pia mater 102. Hemostatis causes edema and ischemia, leading to atrophy of the optic nerve 30 and the formation of glaucomatous cupping 32. This is a possible reason why some glaucoma development continues even after pharmaceutical and/or surgical treatments have reduced intraocular pressure to within what is considered to be the normal range.

In the anterior portion 34 of the eye 8, blood filling of the veins increases. Venous stasis begins in the ciliary body 10 and iris 36, altering the permeability of the blood vessels in the ciliary body 10. Possibly, the physical (fluidity and viscosity) and the biochemical composition of the aqueous humor 38 changes. As a result, damage to the blood/aqueous humor barrier and to the routes of aqueous humor outflow and neurohumoral regulation occurs. Increased resistance to the routes of aqueous humor outflow elevates intraocular pressure.

In the situation where the blood-aqueous barrier and mechanism of autoregulation are preserved and damage of routes of outflow does not occur, primary glaucoma with normal intraocular pressure develops.

The present invention is a proposed treatment for the primary glaucoma described above. The treatment includes resection of the meridional fibers 16 of the ciliary muscle 12. The resection can be performed anywhere along the length of the meridional fibers 16, however, it is preferably performed in front of the ora serrata 44, through the sclera 40, as at 50. Preferably, the resection is performed between the places of attachment 46 of the rectus muscles 48. The areas of the anterior ciliary arteries 74 and long posterior ciliary arteries 90 should be left intact.

If a single cut is made from one rectus muscle attachment point 46 to an adjacent attachment point 46, it is possible that the sclera 40 could separate between those two points, allowing leakage of the vitreous humor 42. Consequently, it is preferred that the resection be performed as a series of regularly spaced cuts 54 in two relatively parallel lines 52, as shown in FIG. 6, to prevent leakage of the vitreous humor 42. Note that the cuts 54 of the two lines 52 should overlap slightly to ensure that all of the meridional fibers 16 are resected. The preferred characteristics of the cuts 54 are that each cut 54 is between about 1 mm and 2 mm in length, most preferably about 1.5 mm, and the cuts 54 are separated by gaps 56 of between about 0.5 mm and 1.5 mm, most preferably about 1 mm. Preferably, the lines 52 are separated by a gap 58 of between about 1 mm and 2 mm.

As a result of this surgical procedure, (a) blood filling of the posterior choroid 26 decreases, (b) hemostasis of the posterior ciliary arteries 90, 100 and their branches disappears, (c) the blood supply to the optic nerve 30 improves, and (d) the blood supply to the ciliary body 10 and iris 36 improves.

Use of laser technology is possible. However, the branches of vessels and nerves that pass through suprachoroidal spaces will not regenerate if cut by a laser. In order to reduce trauma for the proposed surgery, a surgical knife with a diamond blade is preferred.

All other traditional surgical procedures for lowering raised intraocular pressure and improving routes of aqueous humor outflow can be used simultaneously with the proposed method.

Thus it has been shown and described a method for treating primary glaucoma caused by the spasming of the meridional fibers of the ciliary body which satisfies the objects set forth above.

Since certain changes may be made in the present disclosure without departing from the scope of the present invention, it is intended that all matter described in the foregoing specification and shown in the accompanying drawings be interpreted as illustrative and not in a limiting sense.

I claim:

1. A method for treating primary glaucoma caused by spasming of the meridional fibers of the ciliary body of the eye comprising resection of the meridional fibers.

2. The method of claim 1 wherein said resection is performed in front of the ora serrata in between the places of attachment of the four rectus muscles of the eye.

3. The method of claim 1 wherein said resection is performed as two relatively parallel lines of spaced cuts.

4. The method of claim 3 wherein each of said spaced cuts is between approximately 1 mm and approximately 2 mm in length and wherein said cuts are separated by a gap of between approximately 0.5 and approximately 1.5 mm.

5. The method of claim 3 wherein said lines are separated by a gap of between approximately 1 mm and approximately 2 mm.

6. The method of claim 1 wherein said resection is performed using a surgical knife having a diamond blade.
